(19) United States
(12) Patent Application Publication
(10) Pub. No.: US 2009/0246841 A1
(43) Pub. Date: Oct. 1, 2009

(54) Methods and compositions for production of acetaldehyde

(76) Inventors: Andrew C. Jamieson, Cambridge, MA (US); Mark Anthony Vandeyar, Cape Town (ZA)

Correspondence Address: LAHIVE & COCKFIELD, LLP;FLOOR 30, SUITE 3000, ONE POST OFFICE SQUARE, BOSTON, MA, 02109, US

(21) Appl. No.: 12/079,274

(22) Filed: Mar. 26, 2008

(51) Int. Cl. C12P 7/06; C12P 7/24

(52) U.S. Cl. 435/147; 435/161

(57) ABSTRACT

The present disclosure provides methods for producing acetaldehyde from renewable biological resources, for example, from a fermentable substrate, with the advantages of energy efficiency and ease of purification. Particular embodiments, feature the production of acetaldehyde from pyruvate, wherein the pyruvate is generated from various carbon sources (e.g., sugars) by pyruvate-producing microorganisms. The methods comprise culturing a pyruvate-producing microorganism in a culture medium under conditions such that pyruvate is excreted and accumulates extracellularly, resulting in a pyruvate-enriched medium.

### FIELD

The present disclosure is in the field of industrial production of commodity chemicals by biotechnology.

### BACKGROUND

Economic and industrial growth have traditionally relied on inexpensive access to fossil fuel resources such as coal and oil, both as a source of energy and as chemical feedstock for the industrial production of organic chemicals. In recent years, the soaring price of crude oil, and the negative effects of fossil fuels on the environment have encouraged the search for alternative energy sources that will ensure continued economic growth and energy independence, while alleviating the negative environmental effects of traditional fossil fuels. One alternative to fossil fuels that is receiving increasing attention is the production of energy and useful chemicals from plant materials through the process of fermentation.

Acetaldehyde is an important chemical intermediate in the production of numerous organic compounds. These include acetic acid, which is used in the production of vinyl acetate; pyridine and pyridine bases, which are widely used in the manufacture of insecticides, herbicides and pharmaceuticals; pentaerythritol, in the production of explosives; and butylene glycol, in the manufacture of cosmetics. Moreover, in addition to these and other useful organic compounds, acetaldehyde is an immediate precursor of ethanol, which can be used as a clean burning, high-octane alternative to fossil fuels. Over 700 million pounds of acetaldehyde are produced annually in the United States via the catalytic oxidation of ethylene obtained from petrochemicals.

The production of acetaldehyde from biological fermentations would offer significant advantages. Biological fermentation does not require the use of oil or other fossil fuels as a starting material. Acetaldehyde has a low boiling point (21° C.), making it volatile at low temperatures and enabling its separation from a fermentation mixture at ambient temperatures; unlike ethanol, which requires an energy intensive distillation step for its separation. Furthermore, acetaldehyde does not form an azeotrope with water, as does ethanol; simplifying its purification and increasing its yield compared to ethanol. Efficient production of acetaldehyde by fermentation could thereby provide a cost-effective means of producing ethanol through the catalytic conversion of acetaldehyde into ethanol.

Intracellular acetaldehyde is produced during ethanolic fermentation by decarboxylation of pyruvate through the action of the enzyme pyruvate dehydrogenase. The acetaldehyde so produced is rapidly converted to ethanol by reduction, through the activity of the enzyme alcohol dehydrogenase. These enzymes are located in an intracellular multi-subunit enzyme complex, which ensures the conversion of pyruvate to ethanol without substantial release of acetaldehyde. Thus, during ethanolic fermentation, acetaldehyde does not accumulate in the cell.

Production of acetaldehyde through microbial fermentation can be obtained, however, by decreasing alcohol dehydrogenase activity in strains of Zymomonas mobilus either by chemical mutagenesis (Wicker and Zall, Appl. Environ. Microbiol., 53(12): 2815-2820, 1987), or by altering the availability of the enzyme's cofactor, NADH, under differing conditions of oxygen supply; in Lactococcus lactis by overexpression of pyruvate decarboxylase and NADH oxidase (Bongers et al., Appl. Environ. Microbiol., 71(2): 1109-1113, 2005); and in Streptococcus thermophilus, through overexpression of a serine hydroxymethyl transferase gene in a process unrelated to ethanolic fermentation (Chaves et al., Appl. Environ. Microbiol., 68(11): 5658-5662, 2005). Acetaldehyde can also be produced in relatively low yield from Saccharomyces cerevisiae in a process used during World War I to make glycerol for dynamite production (Danner and Braun, Chem. Soc. Rev., 28: 395-405, 1999). In this process, acetaldehyde is fixed using sodium sulfite and can no longer act as a hydrogen acceptor during the ethanol fermentation. These processes thus rely on preventing the reduction of acetaldehyde to ethanol during the fermentation process. A potential problem associated with the foregoing biological methods for production of acetaldehyde is that the molecule is toxic to cells. Because acetaldehyde is inhibitory and toxic to the growth of microorganisms, biological production of acetaldehyde generally requires the isolation of acetaldehyde-tolerant mutants, and results in a generally low level of acetaldehyde production (1-4 g/L culture medium). Accordingly, a great need exists for methods of producing acetaldehyde at high yields (>5 g/L) from renewable energy sources.

### SUMMARY

The present disclosure provides methods for producing acetaldehyde from renewable biological resources, for example, from a fermentable substrate, with the advantages of energy efficiency and ease of purification. Particular embodiments, feature the production of acetaldehyde from pyruvate, wherein the pyruvate is generated from various carbon sources (e.g., sugars) by pyruvate-producing microorganisms. The methods comprise culturing a pyruvate-producing microorganism in a culture medium under conditions such that pyruvate is excreted and accumulates extracellularly, resulting in a pyruvate-enriched medium. The pyruvate-producing microorganism can be optionally separated from the pyruvate enriched medium. A pyruvate decarboxylating agent (e.g., pyruvate decarboxylase; thiamine) is then utilized to decarboxylate the pyruvate and produce a high yield of acetaldehyde. The methods and compositions described herein further provide for the production of products derived from acetaldehyde, i.e., products resulting from processes in which acetaldehyde is a chemical intermediate. Such products include, but are not limited to acetic acid, pyridine and pyridine bases, pentaerythritol, butylene glycol and ethanol.

In one aspect, a method is provided for producing acetaldehyde from a biomass, comprising culturing a microorganism under conditions such that pyruvate is excreted, and extracellularly decarboxylating the pyruvate, such that the acetaldehyde is produced from the biomass. In another aspect, a method is provided for producing a high yield of acetaldehyde, comprising culturing a microorganism under conditions such that pyruvate is excreted as extracellular pyruvate, and contacting the extracellular pyruvate with a pyruvate decarboxylating agent, wherein the pyruvate is decarboxylated by the pyruvate decarboxylating agent to produce a high yield of acetaldehyde. In one aspect, a method is provided for producing acetaldehyde from a fermentable substrate, comprising culturing a microorganism in the presence of the fermentable substrate under conditions such that pyruvate is excreted, and extracellularly decarboxylating the pyruvate, such that the acetaldehyde is produced from the fermentable substrate. In another aspect, a high yield production method is provided for producing acetaldehyde, comprising culturing a microorganism under conditions such that pyruvate is excreted and contacting the extracellular pyruvate with a pyruvate decarboxylase enzyme, wherein the pyruvate is decarboxylated by the pyruvate decarboxylase to produce a high yield of acetaldehyde.

In one embodiment, the pyruvate decarboxylating agent is an enzyme. In another embodiment, the enzyme is pyruvate decarboxylase. In another embodiment, the pyruvate decarboxylase is obtained by recombinant DNA procedures. In another embodiment, the pyruvate decarboxylase is obtained from a genetically modified organism. In another embodiment, the pyruvate decarboxylase is immobilized on a solid support. In yet another embodiment, the pyruvate decarboxylase is obtained from a natural source. In yet another embodiment, the source is a microorganism (e.g., a bacterium, a yeast, a fungus or an alga) or a plant (e.g., a crop plant, for example, maize, potatoes (e.g., sweet potatoes or sweet potato root), manioc, beans, rice (or rice bran) peanuts, sunflower, tomato, upland cotton, tobacco and the like). In one embodiment, the plant is maize. In other embodiments, the plant is sweet potato root (see, for example, Oba and Utitani, J. Biochem., 77 (6): 1205-1212, 1975) or rice bran (see, for example, Rivoal J, et al. Eur J. Biochem., 194(3):791-797, 1990). In other embodiments, the source has been genetically modified to obtain a higher yield of pyruvate decarboxylase. In any of the described embodiments, the pyruvate decarboxylase enzyme is optionally genetically engineered such that enzymatic activity or efficiency is increased to a turnover number at least 20/s.

In certain embodiments, the decarboxylating agent is a chemical (e.g. thiamine, a thiamine analogue or a thiamine-related compound). In these embodiments, the extracellular pyruvate is contacted with thiamine, a thiamine analog, or a thiamine-related compound, whereby the pyruvate is decarboxylated to produce a high yield of acetaldehyde. In additional embodiments, the decarboxylating agent is immobilized on a solid support.

In another embodiment, the acetaldehyde is produced at a yield of at least 5 g/L of culture medium. In another embodiment, the acetaldehyde is produced at a yield of 15 g/L. In another embodiment, the pyruvate accumulates extracellularly to a level of at least 10 g/L of culture medium. In another embodiment, the pyruvate accumulates extracellularly to a level greater than 30 g/L of culture medium.

In one embodiment, the method further comprises separating the acetaldehyde from the pyruvate-enriched culture medium by distillation of the acetaldehyde.

In one embodiment, the microorganism is a naturally occurring microorganism. In another embodiment, the naturally occurring microorganism is a microorganism of the genus Vibrio (formerly Beneckia).

In another embodiment, the microorganism has been genetically modified to excrete elevated levels of pyruvate. In another embodiment, the genetically modified organism contains a blocked pyruvate converting enzyme. In another embodiment, the microorganism is a facultative anaerobe which has been genetically modified to produce elevated levels of pyruvate during anaerobic fermentation. In another embodiment, the microorganism belongs to a genus selected from the group consisting of Zymomonas, Escherichia, Scizophyllum, Nocardia, Psuedomonas, Acinetobacter, Enterococcus, Ralstonia, Corynebacterium, Torulopsis, Saccharomyces, Candida and Debaryomyces. In another embodiment, the microorganism is Saccharomyces cerevisiae. In yet another embodiment, the microorganism is an ethanologenic microorganism which has been engineered to produce elevated levels of pyruvate during anaerobic fermentation. In another embodiment, the microorganism is a non-ethanologenic microorganism which has been engineered to produce elevated levels of excreted pyruvate during anaerobic fermentation.

In a further embodiment, the microorganism is a genetically modified yeast which excretes elevated levels of pyruvate. In another embodiment, the genetically modified yeast is selected from the group consisting of a vitamin auxotroph and a yeast that is not an auxotroph. In another embodiment, the genetically modified yeast is a vitamin auxotroph, which is deficient in enzyme cofactors for pyruvate-converting enzymes. In yet another embodiment, the genetically modified yeast is not an auxotroph.

In a further embodiment, the microorganism is a photosynthetic microorganism. In another embodiment, the microorganism is an alga. In a specific embodiment, the alga is a member of the genus Chlamydomonas (e.g., Chlamydomonas rheinhardtii).

In another aspect, a production method is provided for producing at least 5 g/L of acetaldehyde, comprising culturing a pyruvate-producing microorganism under conditions such that pyruvate is excreted from the microorganism and accumulates extracellularly, resulting in a pyruvate-enriched culture medium; optionally separating the pyruvate-producing microorganism from the pyruvate-enriched culture medium; contacting the pyruvate-enriched culture medium with a pyruvate decarboxylase enzyme, wherein the pyruvate in the pyruvate-enriched medium is decarboxylated by the pyruvate decarboxylase enzyme; and separating the acetaldehyde from the pyruvate-enriched culture medium by distillation, wherein at least 5 g/L of acetaldehyde is produced. In certain embodiments, the microorganism is a member of the genus Vibrio (formerly Beneckia). In additional embodiments, the microorganism is a genetically modified strain of Saccharomyces (e.g., S. cerevisiae) or Torulopsis (e.g., T. glabrata).

In certain embodiments, the microorganism has a blocked pyruvate converting enzyme.

In certain embodiments, the acetaldehyde is used as an intermediate in the synthesis of acetaldehyde-derived compounds, for example, acetic acid, vinyl acetate, pyridine, pentaerythritol, butylene glycol and ethanol. Thus, the present disclosure provides methods for producing ethanol wherein the acetaldehyde produced by any of the aforementioned methods is contacted with a reducing agent, resulting in the reduction of acetaldehyde to ethanol. The reducing agent can be chemical or enzymatic. Thus, in another embodiment, the acetaldehyde is converted to ethanol by the alcohol dehydrogenase enzyme. In additional embodiments, the acetaldehyde is converted to ethanol using a chemical reducing agent. In one embodiment, the chemical reducing agent is sodium borohydride. In other embodiments, the acetaldehyde is converted to ethanol using an industrial catalyst. In another embodiment, the acetaldehyde is converted to ethanol using a transition-metal catalyst.

In any of the methods heretofore disclosed, extracellular decarboxylation of pyruvate can occur in the absence of the microorganism that excreted the pyruvate. For example, a pyruvate-enriched culture medium can be separated from the microorganism prior to decarboxylation of the pyruvate.

Thus, the disclosure includes, but is not limited to, the following numbered embodiments:

1. A method for producing acetaldehyde, comprising:
a) culturing a microorganism under conditions such that metabolism of a carbon source results in excretion of pyruvate into the culture medium; and
b) converting the pyruvate to acetaldehyde.

2. The method of embodiment 1, wherein the yield of acetaldehyde is 5 g/L of culture medium or greater.

3. The method of embodiment 1, further comprising purifying or enriching the pyruvate.

4. The method of embodiment 1, further comprising the step of purifying the acetaldehyde by distillation

5. The method of embodiment 1, wherein the microorganism is a naturally occurring microorganism.

6. The method of embodiment 5, wherein the naturally occurring microorganism is a member of the genus Vibrio.

7. The method of embodiment 1, wherein the microorganism has been genetically modified to excrete pyruvate.

8. The method of embodiment 1, wherein the microorganism has a blocked pyruvate-converting enzyme.

9. The method of embodiment 7, wherein the genetically modified microorganism is Escherichia coli.

10. The method of embodiment 7, wherein the genetically modified microorganism is a yeast.

11. The method of embodiment 1, wherein:
(a) the carbon source is CO₂;
(b) the microorganism fixes CO₂ to a sugar or a starch; and
(c) the sugar or starch is metabolized to pyruvate.

12. The method of embodiment 11, wherein the microorganism is a photosynthetic alga.

13. The method of embodiment 12, wherein the photosynthetic alga belongs to the genus Chlamydomonas.

14. The method of embodiment 13, wherein the photosynthetic alga has disruptions in the genes encoding pyruvate formate lyase and pyruvate decarboxylase.

15. The method of embodiment 1, wherein the pyruvate is converted to acetaldehyde through the enzymatic action of pyruvate decarboxylase.

16. The method of embodiment 15, wherein the pyruvate decarboxylase is obtained from a microorganism that has been genetically altered to overexpress pyruvate decarboxylase.

17. The method of embodiment 1, wherein the pyruvate is converted to acetaldehyde using a thiamine-related compound or a thiamine analogue.

18. The method of embodiment 1, further comprising converting the acetaldehyde to another compound.

19. The method of embodiment 18, wherein the acetaldehyde is converted to ethanol.

### BRIEF DESCRIPTION OF THE FIGURES

FIG. 1 depicts the enzymatic reaction of pyruvate decarboxylation.

### DETAILED DESCRIPTION

The development of a method that improves the efficiency and economics of organic feedstock production via fermentation has significant industrial and commercial potential. Accordingly, the present disclosure provides methods and compositions for producing acetaldehyde from a microorganism, with the advantages of energy efficiency and ease of purification. Acetaldehyde is produced via the formation of excreted pyruvate, which is obtained directly from the microorganism. Conversion of the excreted pyruvate into acetaldehyde is effected by catalysis, e.g., by the action of the enzyme pyruvate decarboxylase. Purification of acetaldehyde is achieved by distillation at temperatures that do not require significant energy utilization.

Before further description of the methods and compositions disclosed herein, certain terms are defined.

### I. Definitions

As used herein, the term “renewable resource” refers to a resource or material consumed or used in a process, which is capable of being replenished by natural processes, preferably at a rate comparable to its rate of consumption or use. Exemplary renewable resources include, but are not limited to, oils, for example from plants or seeds, alcohols, gasses, for example, methane, sugars, carbohydrates, and the like, from natural sources. “Renewable resources” are distinguished from “non-renewable resources” in that “non-renewable resources exist in fixed amounts used at a rate faster than can be replenished (e.g., cannot be re-made, re-grown or regenerated ) by nature. Exemplary “non-renewable resources” include carbon-based non-renewables, for example, fossil fuels or petrochemicals.

As used herein, the term “substrate” refers to a molecule upon which an enzyme acts. Preferably, the substrate binds to an active site within the enzyme, and an enzyme-substrate complex is formed. The substrate is then converted into a product (or products) and released from the active site. The active site is now free to accept another substrate molecule.

Substrates can be “isolated” (i.e., separated from usually associated elements or components) or “purified” (i.e., separated from undesirable elements or components) or can be included as part of a complex material, for example, as part of a “biomass”, “biological waste”, “biodegradable waste” or “biological waste stream”. The term “biomass” as used herein, refers to living or recently dead biological material (e.g., plant and/or animal matter) that can be used as fuel or for industrial production. The term “biomass” typically excludes organic material which has been transformed by biological processes into substances such as petrochemicals. The term “biological waste” refers to waste originating from plant and/or animal materials or sources. The term “biodegradable waste” refers to waste originating from plant and/or animal materials or sources, which may be broken down by other living organisms. The term “biological waste stream” refers to a mixture or flow of waste (e.g., plant and/or animal waste) resulting from or produced by biological processes.

As used herein, the terms “ferment”, “fermenting”, or “fermentation” refer to the process by which cells or microorganisms derive or produce energy from the oxidation of organic compounds such as carbohydrates or sugars. Typically, both energy and organic products (e.g., acids, alcohols, gases, for example, carbon dioxide, and the like) are produced as a result of fermentation processes. Fermentation processes are typically anaerobic, i.e., do not require oxygen, however fermentation does not necessarily have to be carried out in an anaerobic environment. For example, “ethanol fermentation” refers to a form of anaerobic respiration used primarily by yeast when oxygen is not present in sufficient quantity for normal cellular respiration. However, even in the presence of abundant oxygen, yeast generally prefer fermentation to oxidative processes, as long as sugars are readily available.

As used herein, the term “fermentable substrate” refers to a substrate which is utilized by an enzyme or enzymatic process, for example, in a microorganism to form a simpler compound or compounds, e.g., a metabolite. Preferred “fermentable substrates” include biological carbon sources, for example, sugars, celluloses, starches, amino acids, and the like. In certain aspects, the fermentable substrates are used or processed by microorganisms to support growth and/or other key biological processes.

Exemplary “fermentable substrates” include sugars or carbohydrates which can be utilized by microorganisms as an energy source or in biosynthesis.

In certain embodiments, the fermentable substrate is glucose which can be converted by the microorganism into two molecules of pyruvate (or pyruvic acid), which is then used to provide further energy, either aerobically or anerobically. Alternatively, pyruvate can be converted (i.e., decarboxylated) to acetaldehyde using, for example, a pyruvate decarboxylating agent. As used herein, the term “pyruvate decarboxylating agent” refers to a compound or agent, either biological or chemical, capable of catalyzing the conversion of pyruvate to acetaldehyde and carbon dioxide.

As used herein, the term “pyruvate-producing microorganism” refers to a microorganism which is capable of secreting pyruvate into a medium, whereby the pyruvate accumulates extracellularly. In one embodiment, the pyruvate accumulates to an extracellular level of at least about 30 g/L, at least about 40 g/L, at least about 50 g/L, at least about 60 g/L, at least about 75 g/L, at least about 85 g/L, at least about 100 g/L, at least about 125 g/L, at least about 135 g/L, at least about 150 g/L, and at least about 165 g/L of culture medium. In another embodiment, pyruvate accumulates to an extracellular level of at least about, greater than, not more than, greater than or equal to, less than or equal to, or less than about 30 g/L, about 40 g/L, about 50 g/L, about 60 g/L, about 75 g/L, about 85 g/L, about 100 g/L, about 125 g/L, about 135 g/L, about 150 g/L, about 165 g/L, and about 500 g/L of culture medium. Values recited herein as an upper or lower limit are intended to be within the scope of the present disclosure. Moreover, values included and/or intermediate within the values set forth herein are also intended to be within the scope of the present disclosure. For example, pyruvate can accumulate to an extracellular level of at least about (or about) 50 g/L, 51 g/L, 52 g/L, 53 g/L, 54 g/L, 55 g/L, 56 g/L, 57 g/L, 58 g/L, 59 g/L, or 60 g/L of culture medium. Unless otherwise stated, the g/L yield of pyruvate stated herein refers to g/L of pyruvate produced in the culture medium.

In one embodiment, pyruvate can accumulate to an extracellular level of between about 50 g/L to about 60 g/L, between about 30 g/L to about 135 g/L, between about 50 g/L to about 125 g/L, between about 60 g/L to about 100 g/L, between about 75 g/L to about 100 g/L, between about 80 g/L to about 100 g/L, between about 50 g/L to about 500 g/L, between about 25 to about 400 g/L, between about 100 to about 300 g/L, between about 150 to about 200 g/L, between about 50 to about 100 g/L, between about 100 to about 200 g/L, between about 200 to about 300 g/L, between about 300 to about 400 g/L, or between about 400 to about 500 g/L of culture medium. Values and ranges included and/or intermediate within the ranges set forth herein are also intended to be within the scope of the present disclosure. For example, pyruvate can accumulate to an extracellular level of between about 55 g/L to about 57 g/L. Ranges having values recited herein as an upper or lower limit are also intended to be within the scope of the present disclosure.

In exemplary embodiments, the pyruvate-producing microorganism (e.g., bacteria, e.g., E. coli) is cultured to a density of about 10⁴ cells/L, 10⁵ cells/L, about 10⁶ cells/L, about 10⁷ cells/L, about 10⁸ cells/L, or between about 10⁵ cells/L to about 10⁶ cells/L. In other exemplary embodiments, the pyruvate-producing microorganism (e.g., a yeast) is cultured to a density of about 10⁶ cells/L, about 10⁷ cells/L, about 10⁸ cells/L, about 10⁹ cells/L, about 10¹⁰ cells/L, or between about 10⁷ cells/L to about 10⁹ cells/L.

In exemplary embodiments, the pyruvate-producing microorganism (e.g., a bacterium, alga, fungus or yeast) is cultured at or to a biomass of about 0.01 g biomass/L, 0.05 g biomass/L, 0.1 g biomass/L, about 0.5 g biomass/L, about 1 g biomass/L, about 1.5 g biomass/L, about 2 g biomass/L, about 5 g biomass/L, about 10 g biomass/L, about 15 g biomass/L, about 20 g biomass/L, about 50 g biomass/L, about 100 g biomass/L or more. In some embodiments, the microorganism is cultured at or to a biomass of about 0.5 g biomass/L to about 50 g biomass/L. In other embodiments, the microorganism is cultured to a density of about 1-5 g biomass/L, about 5-10 g biomass/L, about 10-20 g biomass/L, about 20-50 g biomass/L, or about 50-100 g biomass/L.

Values and ranges included and/or intermediate within the ranges set forth herein are also intended to be within the scope of the present disclosure. Ranges having values recited herein as an upper or lower limit are also intended to be within the scope of the present disclosure.

In one embodiment, the pyruvate-producing microorganism is a naturally occurring microorganism. Naturally-occurring microorganisms include marine microorganisms of the genus Vibrio (the genus was formerly known as Beneckia), which produce pyruvate aerobically. Naturally occurring microorganisms also include, but are not limited to, Zymomonas, Escherichia, Schizophyllum, Nocardia, Psuedomonas, Acinetobacter, Enterococcus, Ralstonia, Corynebacterium, Torulopsis, Saccharomyces, Candida and Debaryomyces. In one embodiment, the naturally occurring microorganism is Saccharomyces cerevisiae.

In another embodiment, the microorganism is an alga. In a specific embodiment, the alga is a member of the genus Chlamydomonas (e.g., Chlamydomonas rheinhardtii). In additional embodiments, the activity of one or both of the enzymes pyruvate formate lyase (pfl) and pyruvate decarboxylase is inactivated in the alga, by mutation or deletion of the gene(s), by inhibition of mRNA translation, by an inhibitor of enzymatic activity or any other method.

In another embodiment, the microorganism is a yeast vitamin auxotroph which excretes elevated levels of pyruvate. In another embodiment, the microorganism is not a yeast vitamin auxotroph.

In another embodiment, the pyruvate-producing microorganism is a genetically modified microorganism which has been modified to excrete elevated levels of pyruvate. In one embodiment, genetically modified microorganisms include Zymomonas, Escherichia, Schizophyllum, Nocardia, Psuedomonas, Acinetobacter, Enterococcus, Ralstonia, Corynebacterium, Torulopsis, Saccharomyces, Candida and Debaryomyces. In one embodiment, the genetically modified microorganism is Saccharomyces cerevisiae. In another embodiment, the genetically modified microorganism has been engineered to produce elevated levels of pyruvate during anaerobic fermentation. In yet another embodiment, the genetically engineered microorganism is an ethanologenic microorganism which has been engineered to produce elevated levels of excreted pyruvate during anaerobic fermentation. In another embodiment, the genetically modified microorganism is a non-ethanologenic microorganism which has been engineered to produce elevated levels of excreted pyruvate during anaerobic fermentation. In yet another embodiment, the genetically modified microorganism is of the genus Vibrio (formerly known as Beneckia).

As used herein, the term “high yield of acetaldehyde” refers to a yield of acetaldehyde that is produced at a yield of at least about 5 g/L of culture medium. In another embodiment, acetaldehyde is produced at a yield of at least about 5 g/L, at least about 6 g/L, at least about 7 g/L, at least about 8 g/L, at least about 9 g/L, at least about 10 g/L, at least about 11 g/L, at least about 12 g/L, at least about 13 g/L, at least about 14 g/L, at least about 15 g/L, at least about 16 g/L, at least about 17 g/L, at least about 18 g/L, at least about 19 g/L, at least about 20 g/L, at least about 25 g/L, at least about 30 g/L, at least about 35 g/L, at least about 40 g/L, at least about 45 g/L, at least about 50 g/L or at least about 75 g/L of culture medium. In one embodiment, acetaldehyde is produced at a yield of between about 5 g/L to about 75 g/L, between about 7 to about 50 g/L, between about 10 g/L to about 25 g/L, between about 12 g/L to about 20 g/L, between about 14 to about 16 g/L, between about 5 g/L to about 10 g/L, between about 10 g/L to about 14 g/L, between about 12 to about 16 g/L, between about 14 g/L to about 20 g/L, between about 20 to about 25 g/L, between about 25 to about 35 g/L, between about 35 g/L to about 50 g/L, or between about 50 to about 75 g/L of culture medium. Values and ranges included and/or intermediate within the ranges set forth herein are also intended to be within the scope of the present disclosure. For example, acetaldehyde is produced at a yield of between about 11 g/L to about 13 g/L of culture medium. Unless otherwise stated, the g/L yield of acetaldehyde stated herein refers to g/L of acetaldehyde produced in the culture medium. Ranges having values recited herein as an upper or lower limit are also intended to be within the scope of the present disclosure.

The term “genetically-modified microorganism” includes all microorganisms whose genetic material has been altered or modified compared to wild-type. Modification or engineering of such microorganisms can be according to any methodology known in the art or described herein. In certain embodiments, genetically modified microorganisms include those organisms which have been modified to alter the activity of a gene and/or gene product of interest. In one embodiment, genetically modified microorganisms include those which have been modified by targeted modification of a gene or regulatory sequence to cause over expression of the gene of interest. In yet another embodiment, a genetically modified organism includes organisms that have been modified such that a transcription factor leads to the overexpression of a gene of interest.

The disclosed methods can utilize organisms that have been genetically modified to accumulate and/or excrete pyruvate, such as, for example, microorganisms having a blocked pyruvate converting enzyme wherein the blocked pyruvate converting enzyme has been generated by genetic modification. Certain embodiments also utilize organisms that have been genetically modified to produce high levels of pyruvate decarboxylase compared to wild-type and/or pyruvate decarboxylase having higher-than-normal activity (e.g., increased turnover number) compared to the wild-type enzyme. Such are useful for the production of pyruvate decarboxylase used in the conversion of pyruvate to acetaldehyde. Certain embodiments can involve the use of both types of genetically modified organisms.

The term “overexpressed” or “overexpression” includes expression of a gene product (e.g., a pyruvate biosynthetic enzyme) at a level greater than that expressed prior to modification of the microorganism or than that expressed in a comparable microorganism which has not been modified. In certain embodiments, the microorganism can be genetically modified (e.g., genetically engineered) to express a gene product at a level greater than that expressed prior to modification of the microorganism or than that expressed in a comparable microorganism which has not been modified (i.e., to overexpress the gene product).

As used herein, the term “enzymatic activity” or “enzymatic efficiency” is intended to include the ability of a polypeptide to enzymatically convert pyruvate into acetaldehyde. Typically, the activity of a selected polypeptide encompasses the total. enzymatic activity associated with the produced polypeptide, comprising, e.g., the superior substrate affinity of the enzyme, thermostability, stability at different pHs, or a combination of these attributes. In one embodiment, the enzymatic activity or efficiency of the pyruvate decarboxylase enzyme is increased to a turnover number of at least 20/s. In another embodiment, the enzymatic activity or efficiency of the pyruvate decarboxylase enzyme is increased to a turnover number of at least 20/s, 30/s, 40/s, 50/s, 60/s, 70/s, 80/s, 90/s, 100/s, 125/s, 150/s, 175/s, 200/s, 225/s, 250/s, 300/s, 325/s, 350/s, 375/s, 400/s, 450/s, 500/s, 550/s, 600/s, 650/s, 700/s, or 750/s.

As used herein, the term “distillation” refers to a process by which chemical substances are separated based on differences in their volatilities. Typically, heat is applied to a mixture of substances and substances evaporate according to their respective volatilities. Evaporated substances (i.e., vapors) are typically collected via condensation. Distillation can be performed as a batch or continuous process. In “batch distillation,” the composition of the source material or mixture, the vapors of the distilling compounds, and the distillate change during the process. In “continuous distillation”, the source materials, vapors and distillate are kept roughly constant by replenishing the source material and removing fractions of the vapor and/or liquids in the system. In one embodiment, acetaldehyde is distilled without the application of external heat. The term “pH” is intended to include its art-recognized meaning. Typically, the pyruvate decarboxylase enzymes as disclosed herein exhibit maximal decarboxylase activity at a pH of about 4 to about 8, more particularly at a pH of about 5 to about 7, even more particularly at a pH of about 5.5 to about 6.0. In certain embodiments, the pH of the pyruvate-containing medium can be adjusted so that the enzyme has optimal activity (i.e., an “optimal pH”).

In certain embodiments, other conditions of the pyruvate-containing medium, for example, salt concentration, inclusion of protease inhibitors, and the like, may be altered to achieve optimal pyruvate decarboxylase enzyme activity.

As used herein, the term “turnover number” or “k_{cat}” refers to the maximum number of molecules of substrate that an enzyme can covert to product per catalytic site per unit time.

The term “substrate affinity” is intended to include the binding kinetics of an enzyme for a substrate, for example, the K_{M} of the enzyme pyruvate decarboxylase for its substrate pyruvate (or analogue thereof). Typically, the pyruvate decarboxylase enzymes as disclosed herein exhibit a substrate affinity (e.g., for pyruvate) having a K_{M} of about 0.1 mM to about 10 mM (e.g., a K_{M} of about 0.5 mM, about 1 mM or about 5 mM).

The terms “thermal stability” and “thermostability” are used interchangeably and are intended to include the ability of a enzyme (e.g., whether expressed in a cell, present in an cellular extract, cell lysate, or in purified or partially purified form) to catalyze a reaction (e.g., the conversion of pyruvate to acetaldehyde) at least at about 20° C., advantageously at about 25° C. to 35° C., more advantageously at about 37° C. or higher, in particular, at about 50° C. or higher, for example, at least about 60° C. or higher.

The term “ethanologenic” is intended to include the ability of a microorganism to produce ethanol from a carbohydrate as a primary fermentation product. The term is intended to include naturally occurring ethanologenic organisms, ethanologenic organisms with naturally occurring or induced mutations, and ethanologenic organisms which have been genetically modified.

The terms “secrete” and “excrete” are used interchangeably herein to refers to the generation, discharge, or release of a substance (e.g., pyruvate) from a microorganism into the extracellular environment.

As used herein, the term “reducing agent” refers to any chemical agent capable of donating electrons; e.g., to acetaldehyde during its conversion to ethanol.

As used herein, the term “intermediate in the synthesis of acetaldehyde-derived compounds” refers to a compound obtained by chemical conversion of acetaldehyde, including but not restricted to acetic acid, polyvinyl acetate, pyridine and pyridine bases, pentaerythritol, butylene glycol and ethanol.

### II. Microorganisms and Methods for Culturing Microorganisms Such That Pyruvate is Over Produced

The methods and compositions of the present disclosure feature microorganisms, e.g., naturally occurring microorganisms and genetically modified microorganisms, cultured in a manner which results in the production and excretion of a desired product (e.g. pyruvate). The term “genetically modified microorganism” includes a microorganism (e.g., bacterium, yeast, fungus, etc.) which has been genetically altered, modified or engineered such that it exhibits an altered, modified or different genotype and/or phenotype (e.g., when the genetic modification affects coding nucleic acid sequences of the microorganism) as compared to the naturally-occurring microorganism from which it was derived. In one aspect, the activity of a gene product (e.g. an enzyme) is altered when the genetic modification affects sequences encoding the enzyme; in another aspect, the activity of a gene product is altered when the genetic modification affects the level of expression of a gene (e.g. a genetic modification affecting sequences that regulate expression of an enzyme).

The term “genetically modified microorganism” also includes a microorganism that has been modified such that the microorganism has at least one enzyme of a biochemical pathway modified such that pyruvate is over-produced and excreted into the culture medium. Alterations to create such microorganisms can be according to any methodology known in the art or described herein including but not limited to, deregulation of a biosynthetic pathway and/or overexpression of at least one biosynthetic enzyme. In certain embodiments, the genetically modified organism contains an alteration in the activity of a pyruvate converting enzyme. In one aspect, the pyruvate converting enzyme has reduced activity compared to that expressed prior to modification of the microorganism or to that expressed in a comparable microorganism which has not been modified. In another aspect, the pyruvate converting enzyme (e.g. lactate dehydrogenase) has increased activity compared to that expressed prior to modification of the microorganism or to that expressed in a comparable microorganism which has not been modified.

Genetic modification can include, but is not limited to, altering or modifying regulatory sequences or sites associated with expression of a particular gene (e.g., by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site, a promoter, or a transcription terminator, increasing the copy number of a particular gene, modifying proteins (e.g., regulatory proteins, suppressors, enhancers, transcriptional activators and the like) involved in transcription of a particular gene and/or translation of a particular gene product, or any other conventional means of modulating the expression of a particular gene product that is routine in the art (including but not limited to use of antisense nucleic acid molecules, for example, to block expression of repressor proteins).

Additional modifications that can result in altered activity of a gene product (e.g., an enzyme, e.g., a pyruvate converting enzyme) include epigenetic modifications such as DNA methylation, histone modification (e.g., methylation, acetylation, phosphorylation, ubiquitination) and other alterations in chromatin structure.

In another embodiment, the microorganism can be physically or environmentally modified to overexpress a level of gene product greater than that expressed prior to modification of the microorganism or than that expressed in a comparable microorganism which has not been modified. For example, a microorganism can be treated with or cultured in the presence of an agent known or suspected to increase transcription of a particular gene and/or translation of a particular gene product such that transcription and/or translation are enhanced or increased. Alternatively, a microorganism can be cultured at a temperature selected to increase transcription of a particular gene and/or translation of a particular gene product such that transcription and/or translation are enhanced or increased.

In one embodiment, a microorganism is a bacterium. In another embodiment, the bacterium belongs to the genera Zymomonas, Escherichia, Schizophyllum, Nocardia, Psuedomonas, Acinetobacter, Enterococcus, Ralstonia, and Corynebacterium.

In one embodiment, a bacterium is a Gram positive microorganism (e.g., a microorganism which retains basic dye, for example, crystal violet, due to the presence of a Gram-positive wall surrounding the microorganism). In one embodiment, the bacterium is a bacterium belonging to the genus Lactococcus, Enterococcus, Corynebacterium, or Nocardia. In another embodiment, the microorganism is Lactococcus lactis.

In another embodiment, the bacteria is a Gram negative (excludes basic dye) organism. In yet another embodiment, the bacterium belongs to a genus selected from the group consisting of Escherichia, Zymomonas, Ralstonia, Acinetobacter, Psuedomonas, or Vibrio (formerly Beneckia).

In another embodiment, the microorganism is a fungus or a yeast. In another embodiment, the yeast belongs to the genera Torulopsis, Saccharomyces, Candida or Debaryomyces. In another embodiment, the yeast is Saccharomyces (e.g., S. cerevisiae) or Torulopsis (e.g., Torulopsis glabrata).

In another embodiment, the microorganism is an alga (e.g., a green alga or a blue-green alga). In a specific embodiment, the alga is a member of the genus Chlamydomonas (e.g., Chlamydomonas rheinhardtii).

In additional embodiments, the microorganism is an Archaeal microorganism, e.g., a member of the genus Cyanobacter, Mettalosphaera, Sulfolobus, Archaeoglobus, Cenarchaeum, Acidianus or Nitrosopumilus.

An aspect of the present disclosure involves culturing the microorganisms described herein, such that a desired compound (e.g., pyruvate) is produced. The term “culturing” includes maintaining and/or growing a living microorganism of the present disclosure (e.g., maintaining and/or growing a culture or strain). In one embodiment, a microorganism is cultured in liquid medium. In another embodiment, a microorganism is cultured in solid or semi-solid medium. In certain embodiments, a microorganism is cultured in medium (e.g., a sterile, liquid medium) comprising nutrients essential or beneficial to the maintenance and/or growth of the microorganism (e.g., carbon sources or carbon substrate, for example complex carbohydrates such as bean or grain meal, starches, sugars, sugar alcohols, hydrocarbons, oils, fats, fatty acids, organic acids and alcohols; nitrogen sources, for example, vegetable proteins, peptones, peptides and amino acids derived from grains, beans and tubers, proteins, peptides and amino acids derived form animal sources such as meat, milk and animal byproducts such as peptones, meat extracts and casein hydrolysates; inorganic nitrogen sources such as urea, ammonium sulfate, ammonium chloride, ammonium nitrate and ammonium phosphate; phosphorus sources, for example, phosphoric acid, sodium and potassium salts thereof; trace elements, for example, magnesium, iron, manganese, calcium, copper, zinc, boron, molybdenum, and/or cobalt salts; as well as growth factors such as amino acids, vitamins, growth promoters and the like).

Preferably, microorganisms are cultured at a controlled pH. The term “controlled pH” includes any pH which results in production of the desired product (e.g., pyruvate). In one embodiment, microorganisms are cultured at a pH of about 7. In another embodiment, microorganisms are cultured at a pH of between 6.0 and 8.5. The desired pH may be maintained by any number of methods known to those skilled in the art.

Also, microorganisms can be cultured under controlled aeration. The term “controlled aeration” includes sufficient aeration (e.g., oxygen) to result in production of the desired product (e.g., pyruvate). In one embodiment, aeration is controlled by regulating oxygen levels in the culture, for example, by regulating the amount of oxygen dissolved in culture medium. Preferably, aeration of the culture is controlled by agitating the culture. Agitation may be provided by a propeller or similar mechanical agitation equipment, by revolving or shaking the growth vessel (e.g., fermentor) or by various pumping equipment. Aeration may be further controlled by the passage of sterile air or oxygen through the medium (e.g., through the fermentation mixture). In some embodiments, controlling aeration includes lowering oxygen levels sufficiently to support anaerobic fermentation. Optionally, microorganisms are cultured without excess foaming (e.g., via addition of antifoaming agents).

Moreover, microorganisms can be cultured under controlled temperature. The term “controlled temperature” includes any temperature which results in production of the desired product (e.g., pyruvate). In one embodiment, controlled temperatures include temperatures between 15° C. and 95° C. In another embodiment, controlled temperatures include temperatures between 15° C. and 70° C. In other embodiments, temperatures are between 20° C. and 55° C., between 30° C. and 45° C. or between 30° C. and 50° C.

Microorganisms can be cultured (e.g., maintained and/or grown) in liquid medium and preferably are cultured, either continuously or intermittently, by conventional culturing methods such as standing culture, test tube culture, shaking culture (e.g., rotary shaking culture, shake flask culture, etc.), aeration spinner culture, or fermentation. In one embodiment, the microorganisms are cultured in shaker flasks. In another embodiment, the microorganisms are cultured in a fermentor (e.g., a fermentation process). Fermentation processes include, but are not limited to, batch, fed-batch and continuous methods of fermentation. The phrase “batch process” or “batch fermentation” refers to a closed system in which the composition of media, nutrients, supplemental additives and the like is set at the beginning of the fermentation and not subject to alteration during the fermentation, however, attempts may be made to control such factors as pH and oxygen concentration to prevent excess media acidification and/or microorganism death. The phrase “fed-batch process” or “fed-batch” fermentation refers to a batch fermentation with the exception that one or more substrates or supplements are added (e.g., added in increments or continuously) as the fermentation progresses. The phrase “continuous process” or “continuous fermentation” refers to a system in which a defined fermentation medium is added continuously to a fermentor and an equal amount of used or “conditioned” medium is simultaneously removed, preferably for recovery of the desired product (e.g., pyruvate). A variety of such processes have been developed and are well-known in the art.

The phrase “culturing under conditions such that a desired compound (e.g., pyruvate) is produced” includes maintaining and/or growing microorganisms under conditions (e.g., temperature, pressure, pH, aeration, nutrient composition, duration, etc.) appropriate or sufficient to obtain production of the desired compound or to obtain desired yields of the particular compound being produced. For example, culturing is continued for a time sufficient to produce the desired amount of pyruvate. Preferably, culturing is continued for a time sufficient to substantially reach maximal production of pyruvate. In one embodiment, culturing is continued for about 12 to 24 hours. In another embodiment, culturing is continued for about 24 to 36 hours, 36 to 48 hours, 48 to 72 hours, 72 to 96 hours, 96 to 120 hours, 120 to 144 hours, or greater than 144 hours. In another embodiment, culturing is continued for a time sufficient to reach production yields of pyruvate, for example, cells are cultured such that at least about 10 g/L of pyruvate are produced, at least about 10 to 15 g/L of pyruvate (e.g., at least about 10, 11, 12, 13, 14, or 15 g/L pyruvate) are produced, at least about 15 to 20 g/L of pyruvate are produced, at least about 20 to 25 g/L pyruvate are produced, at least about 25 to 30 g/L pyruvate are produced, at least about 30 to 35 g/L pyruvate are produced, at least about 35 to 40 g/L pyruvate are produced, or at least about 40 to 50 g/L pyruvate are produced. In yet another embodiment, microorganisms are cultured under conditions such that a yield of pyruvate, for example, a yield within a range set forth above, is produced in about 24 hours, in about 36 hours, in about 48 hours, in about 72 hours, or in about 96 hours.

In one embodiment, the pyruvate accumulates extracellularly to a level of at least 100 g/L of culture medium or a level of at least 150 g/L of culture medium. In another embodiment, the pyruvate accumulates extracellularly to a level greater than 50 g/L of culture medium, a level greater than 60 g/L of culture medium, a level greater than 100 g/L of culture medium, or a level greater than 125 g/L of culture medium.

The methodology disclosed herein can optionally include a step of recovering a desired compound (e.g., pyruvate). The term “recovering” a desired compound (e.g., pyruvate) includes extracting, harvesting, isolating, enriching or purifying the compound from culture medium. Recovering the compound can be performed according to any conventional isolation or purification methodology known in the art including, but not limited to, treatment with a conventional resin (e.g., anion or cation exchange resin, non-ionic adsorption resin, etc.), treatment with a conventional adsorbent (e.g., activated charcoal, silicic acid, silica gel, cellulose, alumina, etc.), alteration of pH, solvent extraction (e.g., with a conventional solvent such as an alcohol, ethyl acetate, hexane and the like), dialysis, filtration, concentration, crystallization, recrystallization, pH adjustment, lyophilization and the like. For example, a compound (e.g., pyruvate) can be preferentially recovered from culture medium by first removing the microorganisms from the culture (e.g., by centrifugation).

A desired compound can be extracted, isolated or purified such that the resulting preparation is substantially free of other components (e.g., free of media components and/or fermentation byproducts). The language “substantially free of other components” includes preparations of desired compound in which the compound is separated (e.g., purified or partially purified) from media components or fermentation byproducts of the culture from which it is produced. In one embodiment, the preparation has greater than about 80% (by dry weight) of the desired compound (e.g., less than about 20% of other media components or fermentation byproducts), more preferably greater than about 90% of the desired compound (e.g., less than about 10% of other media components or fermentation byproducts), still more preferably greater than about 95% of the desired compound (e.g., less than about 5% of other media components or fermentation byproducts), and most preferably greater than about 98-99% desired compound (e.g., less than about 1-2% other media components or fermentation byproducts).

In an alternative embodiment, the desired compound (e.g., pyruvate) is not purified from the microorganism. For example, the entire culture (or culture supernatant) can be used as a source of product (e.g., crude product). In one embodiment, the culture (or culture supernatant) is used without modification. In another embodiment, the culture (or culture supernatant) is concentrated. In yet another embodiment, the culture (or culture supernatant) is dried or lyophilized.

### III. Pyruvate Converting Enzymes

The production and utilization of pyruvate occupies a central location in a number of cellular metabolic pathways; indeed, pyruvate is the molecular link between the glycolytic pathway and the citric acid cycle. Pyruvate also links glycolysis to lactic acid production in homolactic fermentation, and to production of acetaldehyde (and ultimately ethanol and CO₂) in alcoholic fermentation. In addition, pyruvate serves as a substrate for amino acid biosynthesis and in the anaplerotic production of oxaloacetate, which can enter the citric acid cycle or be used as a substrate for gluconeogenesis.

As a result of pyruvate's central role in cellular metabolism, there are a number of enzymes involved in the synthesis of pyruvate, or that use pyruvate as substrate for the synthesis of other molecules. Such enzymes are designated “pyruvate converting enzymes” for the purposes of this disclosure. Since most biochemical reactions are reversible, enzymes that, under normal intracellular conditions, convert a pyruvate precursor to pyruvate (i.e., an enzyme involved in the synthesis of pyruvate) can, under different conditions (e.g., excess pyruvate) also catalyze conversion of pyruvate to the pyruvate precursor and are thus considered to be pyruvate converting enzymes for the purposes of this disclosure.

Exemplary pyruvate converting enzymes include, but are not limited to, the following. Pyruvate decarboxylase catalyzes the conversion of pyruvate to acetaldehyde and CO₂. Lactate dehydrogenase catalyzes the conversion of lactate to pyruvate. The pyruvate dehydrogenase complex catalyzes the conversion of pyruvate to Acetyl Coenzyme A. Pyruvate carboxylase catalyzes the conversion of pyruvate and CO₂ to oxaloacetate. Aminotransferase (transaminase) catalyzes the conversion of pyruvate to alanine. Pyruvate kinase catalyzes the interconversion of pyruvate and phosphoenolpyruate (PEP). Pyruvate:ferredoxin oxidoreductase is an enzyme found in archaeal cells that catalyzes the conversion of pyruvate to acetyl-Coenzyme A in the absence of NAD⁺. Pyruvate phosphate dikinase is a plant enzyme which simultaneously phosphorylates pyruvate (converting it to PEP) and Pᵢ, using the β and γ phosphoryl groups, respectively, of ATP. Additional pyruvate converting enzymes include, but are not limited to, pyruvate ketoglutarate dehydrogenase and pyruvate formate lyase. For additional details of pyruvate metabolism and pyruvate converting enzymes, see, for example, Voet et al. (2008) “Fundamentals of Biochemistry,” Third edition, John Wiley & Sons, Hoboken, N.J., the disclosure of which is incorporated by reference for all purposes.

In certain embodiments, the activity of a pyruvate converting enzyme is altered such that a cell accumulates, and optionally excretes, pyruvate. Such a cell is said to contain a “blocked pyruvate converting enzyme.” In certain embodiments, inhibition of the expression of a gene encoding an enzyme that utilizes pyruvate as a substrate and/or inhibition of the activity of an enzyme that utilizes pyruvate as a substrate results in a cell containing a blocked pyruvate converting enzyme. Cells having one or more blocked pyruvate converting enzymes can be naturally-occurring, obtained by selection or generated by genetic modification.

Depending on intracellular conditions such as reactant concentrations and equilibrium constants of downstream reactions, an enzyme normally involved in the synthesis of pyruvate can, under appropriate conditions, utilize pyruvate as a substrate and convert it to a different molecule. Thus, any method that inhibits the ability of a pyruvate converting enzyme to catalyze a reaction that utilizes pyruvate as a substrate (i.e., any method that generates a blocked pyruvate converting enzyme) leads to increased pyruvate levels and generally, to excretion of pyruvate. Such methods include, but are not limited to, mutation of a gene(s) encoding a pyruvate converting enzyme, either naturally-occurring or induced. Induced mutations include those resulting from chemical mutagenesis, ionizing radiation, transposable elements and/or recombinant DNA technology, for example. Induced mutations also include changes in the nucleotide sequence of a gene resulting from either naturally-occurring or targeted homologous recombination. Mutations can occur in a coding sequence and/or a regulatory sequence and can comprise insertions, deletions, translocations, and/or inversions of any length.

Additional methods for altering the activity of a pyruvate converting enzyme include introduction, into the cell, of an exogenous polynucleotide comprising sequences encoding a protein that alters the expression of a gene. Such a protein can be, for example, a transcriptional regulator such as a transcriptional repressor of a gene encoding a pyruvate converting enzyme. Alternatively, such a protein can be a transcriptional activator that activates expression of a repressor of a gene encoding a pyruvate converting enzyme. See, for example, U.S. Pat. No. 6,534,261 for exemplary disclosure of transcriptional regulators. In additional embodiments, the exogenous polynucleotide can encode or comprise a RNA molecule, such as, for example, a ribozyme, an antisense RNA, RNAi, microRNA, siRNA, etc. Presence of such RNAs in a cell can result in reduced expression of one or more genes encoding pyruvate converting enzymes, or increased expression of genes encoding proteins that inhibit the expression or activity of a pyruvate converting enzyme.

Certain pyruvate converting enzymes require one or more cofactors for their function. For example, the cofactor thiamine pyrophosphate is required for the activity of pyruvate decarboxylase. Accordingly, alterations (as described above) in the activity or function of any gene involved in the production of any cofactor required by a pyruvate converting enzyme can also result in a blocked pyruvate converting enzyme. Such cofactors include, but are not limited to biotin, nicotinic acid, pyridoxine, thiamine, thiamine pyrophosphate (TPP), and the oxidized and reduced forms of nicotinamide adenine dinucleotide (NAD⁺ and NADH, respectively) and nicotinamide adenine dinucleotide phosphate (NADP⁺ and NADPH, respectively).

Other methods of altering the activity of one or more pyruvate converting enzymes include the use of small organic molecules (e.g., molecules with a molecular weight less than 1000 Da), as are generated, for example, for use in the pharmaceutical industry by methods such as, for example, combinatorial chemistry) and naturally-occurring or engineered substrate analogues.

In addition, enzymes involved in the metabolism of products of pyruvate can, under certain conditions, affect intracellular pyruvate levels. For example, during alcoholic fermentation, pyruvate is converted, via the action of pyruvate decarboxylase (PDC), to acetaldehyde, which, in turn, is converted to ethanol through the action of alcohol dehydrogenase (ADH). Inhibition of alcohol dehydrogenase activity, and/or reduced expression of the ADH gene, can result in accumulation of acetaldehyde. Sufficiently high acetaldehyde concentrations in a cell can drive the PDC-catalyzed reaction in the direction of pyruvate formation, leading to accumulation of pyruvate. Thus, alteration of the levels and/or activity of enzymes other than those that interact directly with pyruvate can also be used to obtain increased intracellular pyruvate levels.

### IV. Methodologies for the High Yield Production of Acetaldehyde

A particular embodiment is a high yield production method for producing acetaldehyde comprising culturing a microorganism under conditions such that pyruvate is produced at a significantly high yield. The phrase “high yield production method”, for example, a high yield production method for producing a desired compound (e.g., for producing acetaldehyde) includes a method that results in production of the desired compound at a level which is elevated or above what is usually obtained with comparable production methods. A high yield production method can result in production of the desired compound at a significantly high yield. The phrase “significantly high yield” includes a level of production or yield which is sufficiently elevated or above what is usual for comparable production methods, for example, which is elevated to a level sufficient for commercial production of the desired product (e.g., production of the product at a commercially feasible cost).

One embodiment, features a high yield production method of producing acetaldehyde that includes culturing a microorganism under conditions such that pyruvate is produced at a level greater than 30 g/L of culture medium. In another embodiment, a high yield production method of producing acetaldehyde includes culturing a microorganism under conditions such that pyruvate is produced at a level greater than 50 g/L of culture medium. In another embodiment, a high yield production method of producing acetaldehyde includes culturing a microorganism under conditions such that pyruvate is produced at a level greater than 60 g/L of culture medium. Yet another embodiment features a high yield production method of producing acetaldehyde that includes culturing a microorganism under conditions such that pyruvate is produced at a level greater than 75 g/L of culture medium.

A further embodiment features a high yield production method for producing a desired compound (e.g., for producing acetaldehyde) that involves culturing a microorganism under conditions such that a sufficiently elevated level of compound is produced within a commercially desirable period of time (e.g., time periods longer than 24 hours), optionally resulting in even higher yields of acetaldehyde being produced. For example, the microorganism may be cultured for about 24 hours, for about 36 hours, for about 48 hours, for about 60 hours, for about 72 hours, for about 84 hours, or for about 96 hours.

Specifically, the methods and compositions disclosed herein further feature a high yield production method for producing a desired compound (e.g., for producing acetaldehyde) that involves culturing a microorganism under conditions such that a sufficiently elevated level of compound is produced within a commercially desirable period of time. An exemplary embodiment features a high yield production method of producing acetaldehyde within a 24 hr time period following the production of pyruvate. For example, to process 10,000 liters of culture medium containing 105 g/L pyruvate over a 24 hr time period, approximately 100 g of PDC (with a molecular weight of approximately 60 kDa and a turnover number of 100/s) can be used.

In additional embodiments, the microorganism that is used for overproduction and excretion of pyruvate also contains a pyruvate decarboxylase gene, either endogenous or exogenous, under the transcriptional control of an inducible promoter. Upon accumulation of sufficient pyruvate in the culture medium, as described above, transcription of the pyruvate decarboxylase gene is induced. Depending on the nature of the inducible promoter, inducing agents include temperature (e.g., heat-shock promoter), metal ions (e.g., metallothionein promoter), hormones, small molecules and carbohydrates (e.g., lac, gal, gus promoters). Optionally, in these embodiments, the pyruvate decarboxylase gene is engineered so that the pyruvate decarboxylase is excreted into the culture medium (e.g., by attaching a signal sequence).

### V. Enzymatic Decarboxylation

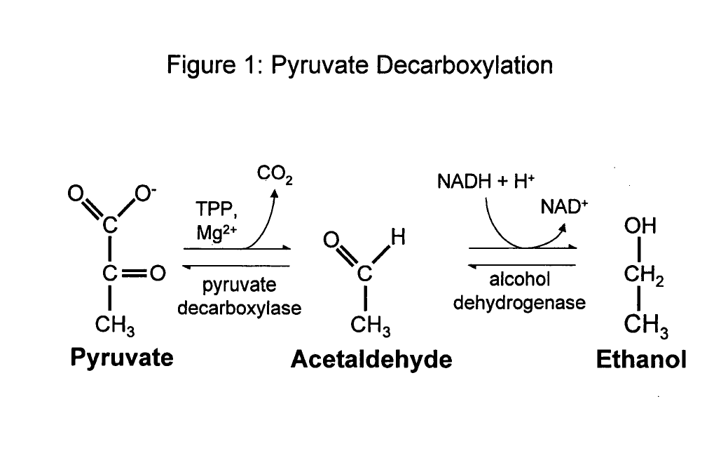

In exemplary embodiments, the pyruvate decarboxylating agent is an enzyme. In certain embodiments, the enzyme is pyruvate decarboxylase, also referred to interchangeably as 2-oxo-acid carboxylase, alpha-ketoacid carboxylase, and pyruvic decarboxylase. Under anaerobic conditions, pyruvate decarboxylase is part of the fermentation process that occurs in microorganisms (e.g., in pyruvate-producing microorganisms) to further produce alcohol. Pyruvate decarboxylase starts this process by converting pyruvate into acetaldehyde and carbon dioxide, after which the acetaldehyde is converted to ethanol. The reactions are depicted schematically in FIG. 1.

As used herein, the term “pyruvate decarboxylase enzyme” is intended to include the enzymes described herein capable of decarboxylating pyruvate into acetaldehyde. By convention, the term “pdc” refers to a pyruvate decarboxylase gene whereas the term “PDC” refers to apdc gene product, i.e., a pyruvate decarboxylase polypeptide or enzyme. The pyruvate decarboxylase enzyme can be naturally occurring (e.g., obtained from a natural source, e.g., a microbe (for example a bacterium, or fungus) or a plant (for example, a crop plant)), obtained from a genetically modified organism, or genetically engineered for improved activity, e.g., pyruvate decarboxylase activity, but also, for example, for improved codon usage, substrate (e.g., pyruvate) affinity, thermal stability, and/or activity at a certain pH. Such a pyruvate decarboxylase enzyme can be an altered or chimeric polypeptide to achieve any of the aforementioned properties. In addition, the polypeptide can further comprise heterologous amino acids, for example, an immunotag for purification or detection, or a signal sequence to promote excretion.

Thus, in certain embodiments, extracellular pyruvate is converted to acetaldehyde enzymatically; e.g., by decarboxylation. This decarboxylation can involve the use of isolated proteins (e.g., isolated enzymes, for example isolated pyruvate decarboxylase). In one embodiment, proteins (e.g., isolated pyruvate decarboxylase) are produced by recombinant DNA techniques and can be isolated from microorganisms of the present disclosure by an appropriate purification scheme using standard protein purification techniques. In another embodiment, proteins are synthesized chemically using standard peptide synthesis techniques.

A cell can be genetically engineered to overexpress an endogenous pyruvate decarboxylase gene. Such methods can involve, for example, promoter insertion (e.g., U.S. Pat. Nos. 6,214,622 and 6,361,972), introduction of exogenous transcription factors (e.g., U.S. Pat. No. 6,534,261), introduction of one or more siRNA molecules into the cell and/or the use of one or more small organic molecules. In additional embodiments, a cell is genetically engineered to express an exogenous pdc gene, such as by expression of a cDNA encoding pdc in the cell, either transiently or stably.

The host cell containing the pdc gene can also be genetically modified to express increased levels of enzyme cofactor, thiamin pyrophoshate (TPP).

An “isolated” or “purified” protein (e.g., an isolated or purified biosynthetic enzyme) is substantially free of cellular material or other contaminating proteins from the microorganism from which the protein is derived, or substantially free from chemical precursors or other chemicals when chemically synthesized. In one embodiment, an isolated or purified protein has less than about 30% (by dry weight) of contaminating protein or chemicals, more preferably less than about 20% of contaminating protein or chemicals, still more preferably less than about 10% of contaminating protein or chemicals, and most preferably less than about 5% contaminating protein or chemicals.

Included within the scope of the present disclosure are proteins or gene products that are encoded by naturally-occurring genes, such as those found in bacteria, yeast, fungi, or plants. Further included within the scope of the present disclosure are proteins or gene products that are encoded by genes which differ from naturally-occurring genes, for example, genes which have nucleic acids that are mutated, inserted or deleted, but which encode proteins substantially similar to the naturally-occurring gene products disclosed herein. For example, it is well understood that one of skill in the art can mutate (e.g., substitute) nucleic acids which, due to the degeneracy of the genetic code, encode an identical amino acid as that encoded by the naturally-occurring gene. Moreover, it is well understood that one of skill in the art can mutate (e.g., substitute) nucleic acids which code for conservative amino acid substitutions. It is further well understood that one of skill in the art can substitute, add or delete amino acids to a certain degree without substantially affecting the function of a gene product as compared with a naturally-occurring gene product, each instance of which is intended to be included within the scope of the present disclosure.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions×100), preferably taking into account the number of gaps and size of said gaps necessary to produce an optimal alignment.

The comparison of sequences and determination of percent homology between two sequences can be accomplished using a mathematical algorithm. A non-limiting example of a mathematical algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to nucleic acid molecules of the disclosure. BLAST protein searches can be performed with the XBLAST program, score=50, wordlength=3 to obtain amino acid sequences homologous to proteins as disclosed herein. To obtain gapped alignments for comparison purposes, Gapped BLAST can be utilized as described in Altschul et al. (1997) Nucleic Acids Research 25(17):3389-3402. When utilizing BLAST and Gapped BLAST programs, the default parameters of the respective programs (e.g., XBLAST and NBLAST) can be used. Another non-limiting example of a mathematical algorithm utilized for the comparison of sequences is the algorithm of Myers and Miller (1988) Comput Appl Biosci. 4:11-17. Such an algorithm is incorporated into the ALIGN program available, for example, at the GENESTREAM network server, IGH Montpellier, FRANCE or at the ISREC server. When utilizing the ALIGN program for comparing amino acid sequences, a PAM120 weight residue table, a gap length penalty of 12, and a gap penalty of 4 can be used.

In another embodiment, the percent homology between two amino acid sequences can be determined using the GAP program in the GCG software package, using either a Blossom 62 matrix or a PAM250 matrix, and a gap weight of 12, 10, 8, 6, or 4 and a length weight of 2, 3, or 4. In yet another embodiment, the percent homology between two nucleic acid sequences can be accomplished using the GAP program in the GCG software package, using a gap weight of 50 and a length weight of 3.

### VI. Chemical Decarboxylation

In certain embodiments, pyruvate is converted to acetaldehyde using a chemical pyruvate decarboxylating agent. Catalytic decarboxylation of pyruvate can be performed by thiamine in solution without enzymatic support (Yatco-Manzo et al, J. Biol. Chem., 234(4): 733-737, 1959), and with chemical analogs of thiamine (Yount and Metzler, J. Biol. Chem., 234(4):738-741, 1959). Recently, it has also been demonstrated that immobilization of thiamine related compounds (2-α-hydroxybenzyl-thiamine pyrophosphate and 2-α-hydroxyethyl-thiamine pyrophosphate) onto a solid support can also be used to efficiently perform the decarboxylation reaction (Stamatis et al, J. Mol. Catal., 267:120-128, 2007). Thus, in certain embodiments, decarboxylation of pyruvate is performed using a chemical catalyst; in additional embodiments, the catalyst is thiamine, a thiamine analog or a thiamine-related compound.

In one embodiment, the chemical agent is thiamine. In another embodiment, the chemical agent is a thiamine-related compound. As used herein, the terms “thiamine related compound” or “thiamine-related compound” refer to a thiamine-related compound which preserves the chemical ability to decarboxylate pyruvate. Thiamine related compounds are described by Stamatis et al, J. Mol. Catal., 267:120-128, 2007, the entire contents of which are incorporated herein by reference. Examples of thiamine related compounds include 2-α-hydroxybenzyl-thiamine pyrophosphate and 2-α-hydroxyethyl-thiamine pyrophosphate. In one embodiment, thiamine related compounds can be immobilized on a solid support to efficiently perform the decarboxylation reaction. In another embodiment, thiamine related compounds are not immobilized onto a solid support. The term “thiamine related compound” includes “thiamine analogs.”

As used herein, the term “thiamine analog” refers to a structural derivative of thiamine that often differs from it by a single element. Thiamine analogs are well known in the art and include, but are not limited to, AMPMT (3(4-amino-2-methyl pyrimidyl-5-methyl)-thiazolium bromide hydrobromide), pyrithiamine (PT), and allithiamine. Thiamine analogs are described by Yatco-Manzo et al, J. Biol. Chem., 234(4): 733-737, 1959 and by Yount and Metzler, J. Biol. Chem., 234(4):738-741, 1959, the entire contents of each of which are expressly incorporated herein by reference.

The methods and compositions disclosed herein are further illustrated by the following examples which should not be construed as limiting. The contents of all references, patents and published patent applications cited throughout this application are hereby incorporated by reference.

### EXAMPLES

Depending on the organism, and the conditions under which sugar (or another carbon source) is fermented (or otherwise metabolized), pyruvate can be obtained as an excreted product in the culture medium. Several species of microorganisms, including marine microorganisms (Ruby and Nealson, Applied Environ. Microbiol., 34(2):164-169, 1977, incorporated by reference), are capable of excreting large amounts of pyruvate during fermentation, altering the balance between the amount of sugar that is converted into pyruvate and the amount of sugar that is utilized for the production of macromolecules. It is also possible to engineer microorganisms such as E. coli and yeast to excrete pyruvate. See, for example, Causey et al., Proc. Nat'l Acad. Sci U.S.A., 101:2235-2240, 2004; and Schmitt and Zimmermann, J. Bacteriol., 151(3): 1146-1152, 1982 (both incorporated by reference). In the latter case, excretion of pyruvate can result in extracellular pyruvate levels of 135 g/L (van Maris et al., Appl. Environ. Microbiol., 70(1): 159-166, 2004, incorporated by reference). Moreover, as described in Example 2, the amount of sugar that is converted into excreted pyruvate can be controlled by utilizing, for example, vitamin auxotrophs, which are inhibited in the subsequent conversion of pyruvate into additional fermentation products. See, for example, Chen and Lun, Appl. Microbiol. Biot., 57(4): 451-459, 2001 (incorporated by reference).

Extracellular pyruvate is converted to acetaldehyde by a pyruvate decarboxylating agent, either chemical or enzymatic. In one embodiment, an easily obtainable source of pyruvate decarboxylase is utilized, such as enzyme purified from yeast or another appropriate microorganism, e.g., Zymomonas mobilis (Swings and De Ley, Bacteriol. Rev., 41:1-46, 1977, incorporated by reference). For conversion of pyruvate to acetaldehyde, this enzyme can be added directly to the culture medium, or added to the culture medium after separation of the pyruvate-excreting cells from the culture medium, or by separation of pyruvate from the culture medium through the use of a membrane which excludes the presence of larger molecules, such as proteins.

In yet other embodiments, the pyruvate decarboxylase is obtained through laboratory manipulation of an appropriate microorganism through recombinant DNA techniques, including production of the enzyme from cloned DNA encoding pyruvate decarboxylase contained within an expression vector. See, for example., Raj et al., Appl. Environ. Microbiol., 68(6): 2869-2876, 2004, incorporated by reference. Optionally, expression of a cloned DNA encoding pyruvate decarboxylase is under the control of a chemical inducer, such as IPTG, or another compound. In another embodiment, genetic manipulations result in the conversion of wild-type pyruvate decarboxylase to an artificial version of the enzyme obtained by alteration of its naturally occurring amino acid sequence, in order to facilitate its cloning or alter its enzymatic properties.

A chemical catalyst, such as a compound related to the enzyme's cofactor, which will catalyze the conversion freely in solution, can also be used to obtain acetaldehyde (Yount and Metzler J. Biol. Chem., 234(4): 738-41, 1959, incorporated by reference). Such a catalyst can be used in solution or affixed to a solid support.

According to the present disclosure, acetaldehyde is purified as a distillate from the fermentation medium, requiring only that the medium is maintained at a temperature at or above the boiling point of acetaldehyde (21° C.).

In another embodiment, acetaldehyde is obtained by fixing acetaldehyde with sodium sulfite in a continuous manner by aeration of the culture and exhausting gas from the culture through a solution of sodium sulfite.

Acetaldehyde, produced according to the disclosed methods, can be utilized in the production ethanol by enzymatic conversion, through the use of a chemical reducing agent such as sodium borohydride, or by using a metal-containing catalyst. Thus, the methods and compositions disclosed herein provide the means to produce ethanol obtained from the excreted pyruvate via decarboxylation of the pyruvate and reduction of the acetaldehyde so produced.

### Example 1

### Production of Excreted Pyruvate From Marine Microorganisms

According to the present disclosure, microorganisms which have the ability to excrete pyruvate, i.e., pyruvate-producing microorganisms, are utilized. Marine microorganisms, such as those of the genus Vibrio (formerly Beneckia), are capable of secreting pyruvate into the growth medium when cultivated in the presence of glucose or several other sugars, as a source of carbon (Ruby and Nealson, Applied Environ. Microbiol., 34(2):164-169, 1977). The conversion of sugar to secreted pyruvate under aerobic conditions occurs rapidly and at a high efficiency. Once the carbon source has been depleted, the microorganisms subsequently utilize the excreted pyruvate for further growth.

Thus, cultures of the genus Vibrio are grown under aerobic conditions until depletion of a carbon source. Sugar levels can be measured in the culture using, e.g., a glucostat (Ruby & Nealson, supra). The cells are then optionally separated from the pyruvate-enriched growth medium.

For example, a starter culture is initially pre-grown in a complex medium and subsequently added to a fermentation medium. The fermentation medium comprises, for example, marine water or artificial sea water containing 0.4M NaCl, 0.1M MgSO₄-7H₂O, 0.02M KCl and 0.02M CaCl₂-2H₂O combined with a buffered medium containing the sugar(s). Once the carbon source (i.e., sugar) in the fermentation medium is depleted, the cells are recovered from the fermentation medium containing the excreted pyruvate, and can be reused for further batch conversion of sugar to pyruvate.

### Example 2

### Production of Excreted Pyruvate from Other Microorganisms

Other microorganisms, such as Torulopsis glabrata, Candida lipolytica, and Saccharomyces cerevisiae, are also utilized for production of acetaldehyde according to the methods and compositions disclosed herein. Genetically-modified versions of these microorganisms are capable of excreting pyruvate when grown in the presence of glucose, and the excreted pyruvate can account for the majority of the substrate carbon metabolized (van Maris et al., Appl. Environ. Microbiol., 70(1): 159-166, 2004, incorporated by reference). Studies have suggested that a decrease in the activity of enzymes responsible for the normal catabolism of pyruvate can be responsible for the build-up and excretion of pyruvate (Schmitt and Zimmermann, J. Bacteriol, 151(3): 1146-1152, 1982, incorporated by reference). This is further supported by the observation that the strains of Torulopsis, with the highest pyruvate producing ability, are also multi-vitamin auxotrophs (Chen and Lun, Appl. Microbiol. Biot., 57(4): 451-459, 2001, incorporated by reference). Thiamine is a cofactor of pyruvate dehydrogenase (PDH) and of pyruvate decarboxylase (PDC). Nicotinic acid, biotin and pyridoxine are cofactors of PDH, pyruvate carboxylase (PC) and transaminase. As the multivitamin auxotrophic strains of Torulopsis are unable to synthesize these four vitamins; under conditions of starvation for these vitamins, the cells exhibit decreased activity of PDH, PDC, PC and transaminase, which results in the accumulation and excretion of pyruvate.

A starter culture of any of the above mentioned auxotrophic microorganisms can be grown in a complex medium containing the vitamins thiamine, nicotinic acid, biotin and pyridoxine. The starter culture is then used to inoculate a suitable minimal culture medium containing the necessary salts, an adequate carbon source (e.g., a sugar) but lacking one or more of the vitamins thiamine, nicotinic acid, biotin and pyridoxine. Once the carbon source in the medium is depleted, the cells are recovered from the growth medium containing the excreted pyruvate, and can be reused for further batch conversion of sugar to pyruvate.

### Example 3

### Production of Pyruvate from Engineered Microorganisms

Further provided are strains engineered, by genetic manipulation, to optimize the excretion of pyruvate (Causey et al., Proc. Natl. Acad Sci. USA 101(8):2235-40.2004 Chen and Lun, Appl. Microbiol. Biot., 57(4): 451-459, 2001, van Maris et al., Appl. Environ. Microbiol., 70(1): 159-166, 2004, all incorporated by reference). The engineered strains are obtained using established mutagenesis and genetic screening techniques, or recombinant DNA technology, to disrupt genes by processes such as homologous recombination or non-homologous end-joining.

For example, when an ethanologenic microorganism, such as Zymomonas mobilis (Swings and De Ley, Bacteriol. Rev., 41:1-46, 1977, incorporated by reference), is grown under anaerobic conditions, pyruvate is converted to acetaldehyde by the enzyme pyruvate decarboxylase (PDC). Therefore the disruption of the gene encoding the PDC enzyme in such a microorganism results in the accumulation and excretion of pyruvate into the growth medium when the mutant is grown under anaerobic conditions. Similarly in non-ethanologenic anaerobic fermentations the disruption of genes whose products are responsible for the metabolism of pyruvate can also be disrupted to increase the yield of pyruvate from fermentable sugar sources.

In this embodiment, the genetically modified cells can only metabolize pyruvate under aerobic conditions. Thus, cultures of the engineered stains are grown in a complex medium under aerobic conditions to generate a starter culture. The starter culture is then used to inoculate a suitable minimal medium containing the necessary salts and an adequate carbon source (e.g., a sugar). At this point, imposition of anaerobic conditions results in the accumulation and excretion of excess pyruvate into the growth medium. Once the carbon source in the fermentation medium is depleted, the cells can be recovered from the growth medium containing the excreted pyruvate, and can be reused for further batch conversion of sugar to pyruvate.

### Example 4

### Pyruvate Production by Photosynthetic Microorganisms

The unicellular green alga Chlamydomonas reinhardtii is a photosynthetic organism that is also capable of performing fermentative metabolism similar to that of the mixed acid fermentations of bacteria, such as Escherichia coli. Growing the alga under optimal conditions, including light, CO₂, O₂ and mineral salts allows the alga to grow and behave as a plant. Under anaerobic conditions, the alga produces a complex mixture of fermentation products, including formate and ethanol (Gfeller and Gibbs, Plant Physiol., 75(1): 212-218, 1984, incorporated by reference). The accumulation of these two metabolic products is known to be the result of the activities of two pyruvate-utilizing enzymes, pyruvate formate lyase (PFL) and pyruvate decarboxylase (PDC) (Hemschemeier and Happe, Biochem Soc Trans. 33(1): 39-41, 2005, incorporated by reference). Thus, algae have the ability to fix CO₂ to a sugar or a starch, and to metabolize the sugar or starch to pyruvate.

In one embodiment, genetic modification of the alga to produce pyruvate results from disruption of the pfl and pdc genes, such that when the alga is transferred from aerobic to anaerobic culture conditions, excess pyruvate is excreted into the culture medium. Additionally, the activities of other enzymes involved in pyruvate metabolism can optionally be manipulated according to the methods described herein to enhance the yield of pyruvate.

### Example 5

### Decarboxylation of Pyruvate to Acetaldehyde

In an exemplary embodiment, the conversion of excreted pyruvate to acetaldehyde and carbon dioxide is accomplished using the enzyme pyruvate decarboxylase (PDC). PDC has been obtained from a number of eukaryotic organisms, including yeast and plants, as well as animals, and it can also be obtained from several ethanologenic microorganisms, for example, from species of the genus Zymomonas. Since several versions of the enzyme have been cloned and characterized, the enzyme can also be conveniently overexpressed in a suitable host, such as E. coli, to obtain large quantities of the enzyme.

The PDC can be added directly to the growth medium containing the excreted pyruvate; alternatively, the PDC can be immobilized on a solid support, in which case it can be re-used in the processing of multiple batches of growth medium containing excreted pyruvate.

### Example 6

### Purification of Acetaldehyde

The present methods and compositions provide an economical means for purifying acetaldehyde. Since the boiling point of acetaldehyde is 21° C., it is released as a volatile gas during the decarboxylation process if the growth medium containing the excreted pyruvate is maintained at a temperature equal to or higher than the boiling point of acetaldehyde. Purified acetaldehyde can be recovered by condensation of the released vapor. The CO₂, which is a by-product of pyruvate decarboxylation, can be easily separated from the acetaldehyde as it will not condense under these conditions.

If the purified acetaldehyde is to be further processed to ethanol, the disclosed methods allow one to avoid the problems of ethanol's high boiling point (requiring more energy-intensive distillation procedures) and the tendency of ethanol to form azeotropic mixtures with water, which makes its purification more difficult than that of acetaldehyde, which does not form azeotropes with water.

### Example 7

### Reduction of Acetaldehyde to Ethanol

Ethanol can be produced from the acetaldehyde made according to the methods disclosed herein by a single chemical reduction step. In one embodiment, an enzyme such as alcohol dehydrogenase is used to perform the reaction. The enzyme-catalyzed step requires NADH as a cofactor, which is oxidized to NAD⁺. The process therefore requires replenishment of NADH for the reaction to continue.

In another embodiment, a readily available chemical reducing agent such as sodium borohydride, is used to reduce the acetaldehyde. Sodium borohydride can be added directly to the acetaldehyde, to produce ethanol, borate and hydrogen. Hydrogen, which can also be used as a source of energy, is released as a gas from the mixture.

Alternatively, a metallic catalyst, containing platinum or another suitable metal, can be used to convert the acetaldehyde to ethanol.

### Equivalents

Those skilled in the art will recognize, or be able to ascertain using no more than routine experimentation, many equivalents to the specific embodiments and methods described herein. Such equivalents are intended to be encompassed by the scope of the following claims.

What is claimed is:

1. A method for producing acetaldehyde, comprising:
a) culturing a microorganism under conditions such that metabolism of a carbon source results in excretion of pyruvate into the culture medium; and
b) converting the pyruvate to acetaldehyde.

2. The method of claim 1, wherein the yield of acetaldehyde is 5 g/L of culture medium or greater.

3. The method of claim 1, further comprising purifying or enriching the pyruvate.

4. The method of claim 1, further comprising the step of purifying the acetaldehyde by distillation.

5. The method of claim 1, wherein the microorganism is a naturally occurring microorganism.

6. The method of claim 5, wherein the naturally occurring microorganism is a member of the genus Vibrio.

7. The method of claim 1, wherein the microorganism has been genetically modified to excrete pyruvate.

8. The method of claim 1, wherein the microorganism has a blocked pyruvate-converting enzyme.

9. The method of claim 7, wherein the genetically modified microorganism is Escherichia coli.

10. The method of claim 7, wherein the genetically modified microorganism is a yeast.

11. The method of claim 1, wherein:
(a) the carbon source is CO₂;
(b) the microorganism fixes CO₂ to a sugar or a starch; and
(c) the sugar or starch is metabolized to pyruvate.

12. The method of claim 11, wherein the microorganism is a photosynthetic alga.

13. The method of claim 12, wherein the photosynthetic alga belongs to the genus Chlamydomonas.

14. The method of claim 13, wherein the photosynthetic alga has disruptions in the genes encoding pyruvate formate lyase and pyruvate decarboxylase.

15. The method of claim 1, wherein the pyruvate is converted to acetaldehyde through the enzymatic action of pyruvate decarboxylase.

16. The method of claim 15, wherein the pyruvate decarboxylase is obtained from a microorganism that has been genetically altered to overexpress pyruvate decarboxylase.

17. The method of claim 1, wherein the pyruvate is converted to acetaldehyde using a thiamine-related compound or a thiamine analogue.

18. The method of claim 1, further comprising converting the acetaldehyde to another compound.

19. The method of claim 18, wherein the acetaldehyde is converted to ethanol.
